(19) United States
(12) Patent Application Publication
(10) Pub. No.: US 2010/0223706 A1
(43) Pub. Date: Sep. 9, 2010

(54) WELDING HELMET AUDIO COMMUNICATION SYSTEMS AND METHODS WITH BONE CONDUCTION TRANSDUCERS

(75) Inventors: William Joshua Becker, Manitowoc, WI (US); Kyle Andrew Pfeifer, Appleton, WI (US)

Correspondence Address: FLETCHER YODER (ILLINOIS TOOL WORKS INC.), P.O. BOX 692289, HOUSTON, TX, 77269-2289, US

(73) Assignee: Illinois Tool Works Inc., Glenview, IL (US)

(21) Appl. No.: 12/648,543

(22) Filed: Dec. 29, 2009

Related U.S. Application Data
(60) Provisional application No. 61/176,523, filed on May 8, 2009.
(60) Provisional application No. 61/157,003, filed on Mar. 3, 2009.

(51) Int. Cl. A61F 9/06; H04R 25/00

(52) U.S. Cl. 2/8.2; 381/151

(57) ABSTRACT

A system for welding communication that includes a welding headgear and a bone conduction transducer (BCT) disposed in or on the welding headgear is provided. The BCT facilitates communication of signals to a welding operator via bone conduction. The bone conduction facilitates the transmission of sound directly to an inner ear of the welding operator.

### CROSS REFERENCE TO RELATED APPLICATIONS

This application is a Non-Provisional patent application of U.S. Provisional Patent Application No. 61/176,523, entitled “Helmet Headgear Audio Playback Communications System Using Bone Conduction Audio Transducers”, filed May 8, 2009, and U.S. Provisional Application No. 61/157,003, entitled “Helmet Speaker System Using Bone Conduction Audio Transducers”, filed Mar. 3, 2009, which is herein incorporated by reference.

### BACKGROUND

Embodiments of the present invention relate generally to welding helmet audio communication systems, and, more particularly, to welding systems and methods that incorporate one or more bone conduction transducers.

Welding is a process that has become increasingly ubiquitous in all industries. While such processes may be automated in certain contexts, a large number of applications continue to exist for manual welding operations, which often require an operator to wear protective gear, such as a welding helmet and earplugs, to protect the welder from the harsh welding environment. Such protection, particularly the earplugs, typically limits the ability of the operator to hear noises in the surrounding environment. Additionally, welders often wear traditional headphones with or without the earplugs to listen to music, weld instruction, and so forth. Unfortunately, volumes on such headphones may be set quite loud for the welder to hear the signal, particularly when used with ear plugs. Traditional headphones may affect the ability of the welder to hear the noises from the arc, which are often useful in determining the quality of the weld. Moreover, where there is a need for the welder to communicate with remote operators or devices during a weld, traditional microphones have been inadequate due to high background noise from the welding environment. Accordingly, there exists a need for improved welding helmet audio communication systems.

### BRIEF DESCRIPTION

In a first embodiment, a system for welding communication includes welding headgear and a bone conduction transducer (BCT) disposed in or on the welding headgear to facilitate communication of signals to a welding operator via bone conduction, wherein the bone conduction facilitates the transmission of sound directly to sound processing anatomies within the ear of the welding operator.

In another embodiment, a system for welding communication includes welding headgear and a BCT disposed in or on the welding headgear to facilitate communication of signals via bone conduction from a welding operator to one or more external devices or operators, wherein the bone conduction facilitates the transmission of sound from a bone structure of the welding operator to the BCT.

In a further embodiment, a system for welding communication includes a welding headgear including a headband and a comfort cushion. The system also includes a BCT embedded in the comfort cushion of the welding headgear and configured to receive audio signals from one or more bones of a welding operator via bone conduction and/or to facilitate the transmission of sound directly to sound processing anatomies within the ear of the welding operator.

### DRAWINGS

These and other features, aspects, and advantages of the present invention will become better understood when the following detailed description is read with reference to the accompanying drawings in which like characters represent like parts throughout the drawings, wherein:

FIG. 1 is a diagrammatical illustration of an exemplary welder wearing a helmet adapted for use with one or more BCTs;

FIG. 2 illustrates a BCT integrated into the welding helmet of FIG. 1 that wirelessly transmits and receives information in accordance with embodiments of the present invention;

FIG. 3 illustrates exemplary circuitry that may be associated with the BCT in the welding helmet to facilitate the receiving and transmitting of data;

FIG. 4A is a diagrammatical top view of a head of a welder wearing headgear with a single BCT disposed in front of a securement headband in accordance with aspects of the present invention;

FIG. 4B is a diagrammatical top view of a head of a welder wearing headgear with two BCTs disposed in front of a securement headband in accordance with aspects of the present invention;

FIG. 4C is a diagrammatical top view of a head of a welder wearing headgear with a single BCT disposed behind a securement headband in accordance with aspects of the present invention;

FIG. 4D is a diagrammatical top view of a head of a welder wearing headgear with two BCTs disposed behind a securement headband in accordance with aspects of the present invention;

FIG. 4E is a diagrammatical top view of a head of a welder wearing headgear with a single BCT disposed in a comfort cushion at a back of the headgear in accordance with aspects of the present invention;

FIG. 4F is a diagrammatical top view of a head of a welder wearing headgear with two BCTs disposed in a comfort cushion at a back of the headgear in accordance with aspects of the present invention;

FIG. 5 is a rear perspective view of exemplary welding headgear that includes BCT electronics mounted onto a back of the headgear;

FIG. 6 illustrates exemplary welding headgear including a modified headband that includes a flexible extension with an opening configured to receive safety goggles;

FIG. 7 illustrates an exemplary mechanism that includes an exemplary hinge structure for mounting a BCT to welding headgear in accordance with aspects of the present invention;

FIG. 8 illustrates an exemplary mechanism that includes a modified headband of the welding headgear that includes a torsion spring mechanism in accordance with aspects of the present invention;

FIG. 9 illustrates an embodiment of the present invention in which the BCTs may be mounted above a lens cartridge inside a shell of a welding helmet;

FIG. 10 is a perspective view of an exemplary embodiment of the welding headgear including the BCT embedded within the comfort cushion;

FIG. 11A illustrates a diagrammatical cross sectional view of an exemplary comfort cushion with an embedded BCT when the headgear is not positioned on a user;

FIG. 11B illustrates a diagrammatical cross sectional view of an exemplary comfort cushion with an embedded BCT when the headgear is positioned on a user;

FIG. 12A illustrates an exemplary spring biased BCT contact mechanism with a dome for contacting a head of a user;

FIG. 12B illustrates an exemplary spring biased BCT contact mechanism with a padded disk for contacting a head of a user;

FIG. 12C illustrates an exemplary lever based BCT contact mechanism for contacting a head of a user;

FIG. 12D illustrates an exemplary spring biased BCT contact mechanism with a movable BCT housing for contacting a head of a user;

FIG. 12E illustrates an exemplary lever based BCT contact mechanism that includes two fixed ends;

FIG. 12F illustrates an exemplary lever based BCT contact mechanism that includes a pivot point contact system;

FIG. 13 illustrates a bottom perspective view of an embodiment of the welding headgear with one or more BCTs attached to the headgear via flexible bands;

FIG. 14 is a cutaway view of the BCT attachment mechanism of FIG. 13 illustrating the flexibility of the bands in accordance with aspects of the present invention; and

FIG. 15 is a perspective view of an embodiment of a removable accessory for a welding helmet that includes an embedded BCT and associated electronics.

### DETAILED DESCRIPTION

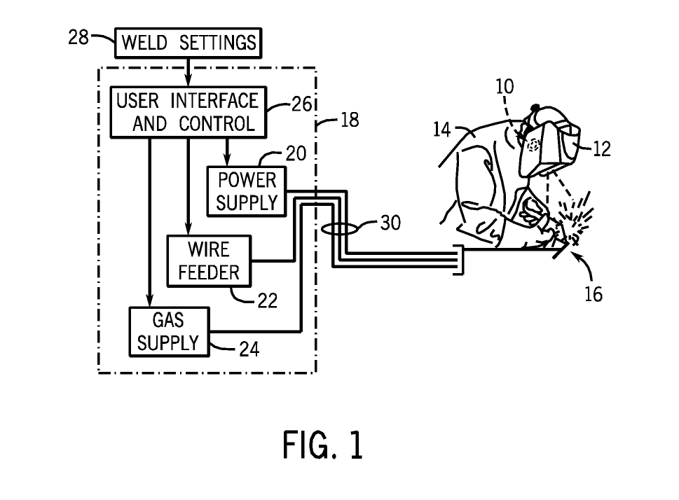

FIG. 1 illustrates a BCT 10 positioned in or on a welding helmet 12 worn by a welder 14. The BCT 10 is designed to transmit sound directly to the inner ear via conduction through areas (e.g., temple, jawbone) of the head of the welder 14. Such a feature may have the effect of maintaining the availability of the ear canal such that the welder 14 may still hear environmental sounds, and so forth, through the ear canal. For instance, the welder 14 may receive audio communications (e.g., weld instructions) via the BCT 10 while maintaining the ability to monitor the sound of the welding operation 16. Furthermore, the welder 14 may wear ear protection, such as ear plugs, while welding and still have the ability to receive audio communication via the BCT 10.

In the illustrated embodiment, one BCT 10 is shown mounted inside the shell of the welding helmet 12. However, in further embodiments any suitable number of BCTs may be located in or on the welding helmet 12 or any other welding apparatus (e.g., goggles). It should be noted that as used herein, the welding helmet includes a shell and welding headgear. During use, the welding headgear is configured to fit onto the head of the welder and receive the shell. The one or more BCTs 10 may be incorporated into or on the welding helmet in a variety of ways. For example, the BCT may be associated with the shell of the helmet. Specifically, the BCT may be clipped onto the helmet shell, held against the helmet shell by a variety of assemblies, or incorporated into the helmet shell with a removable device (e.g., lens cartridge), among other ways. In some embodiments, the BCT may be incorporated within the helmet shell such that the BCT is protected from the harsh conditions of the welding environment. However, in other embodiments, the BCT may be encapsulated in a protective casing and located external to the helmet shell. Indeed, any suitable placement of the BCTs on the helmet shell may enable the transmission of audio communication to the welder 14. That is, incorporation of the BCT into the shell of the welding helmet may allow the BCT to function as a speaker during use.

Still further, in other embodiments, the BCT may be incorporated into the welding headgear at a variety of suitable mounting points. For example, the BCT may be mounted to the headband of the welding headgear to contact one or more of the bones (e.g., cheek bone) of the welder. For further example, the BCT may be attached to the headgear via a flexible extension that is configured to adjust to a variety of positions to accommodate multiple head sizes and shapes. In any of the embodiments in which the BCT is mounted to the welding headgear, the BCT may be partially embedded in foam or another suitable material to isolate the BCT from the vibrations of the headgear. Indeed, any suitable placement of the BCTs on the welding headgear may enable the unidirectional or bidirectional audio communication to and/or from the welder 14. That is, incorporation of the BCT into the headgear of the welding helmet may allow the BCT to function as a speaker and/or a microphone during use. It should further be noted that the BCT may also be incorporated into a comfort cushion that is attached to the back of the welding headgear and configured to contact the back of the head of the welder. In such embodiments, the embedded BCT may be located in a removable accessory (e.g., a comfort cushion) that is removably securable to the headband of the headgear.

It should be noted that presently contemplated embodiments that integrate the BCT with the welding helmet may be employed in conjunction with other technology designed for use in high noise environments. For example, the welding helmet with an integrated BCT may be used with noise suppression technology that filters out specific noises (e.g., background noise) while amplifying and transmitting other audio (e.g., voices). For further example, the noise suppression technology may filter out specific bandwidths while amplifying and transmitting other bandwidths. In presently contemplated embodiments, the welding helmet with the BCT may be used to amplify and transmit the filtered audio directly to the inner ear of the welder via bone conduction. That is, the BCT integrated welding helmet may further include a microphone and a processor that record noises in the welding environment and filter such noise to isolate the voices. Such isolated voices from the environment may then be amplified and transmitted to the welder via the BCT.

The welding operation 16 will typically be powered and controlled by a welding system 18, which interfaces with a welding torch manipulated by the welder 14. In the illustrated embodiment, welding system 18 includes a power supply 20, a wire feeder 22, and a gas supply 24 that provide power, welding wire, and gas, respectively, for the welding operation 16. In other embodiments, the welding system 18 may include more or fewer components based on the type of welding operation selected (e.g., MIG welding, TIG welding, stick welding). The power supply 20 may include, but is not limited to, inverter circuitry, or more generally converter circuitry that produces DC or AC output and that may operate in constant current or constant voltage regimes, pulsed regimes, or other known welding regime. The wire feeder 22 provides a controllable feed of welding wire, such as for metal inert gas (MIG) operations. The gas supply 24 provides shielding gas for such operations when appropriate. As will be appreciated by those skilled in the art, certain of these components may be present in some system types, but absent from others (e.g., gas used for MIG systems, but not for stick or TIG welding, etc.).

A user interface 26 allows the welder 14 to control the welding parameters, such as current, voltage, wire feed speed, specific programmed welding regimes, and so forth. For instance, the user may input desired weld settings 28 into the welding system 18. These settings may include but are not limited to current level, voltage level, welding process, and so forth. Accordingly, the welding system 18 may include memory circuitry to facilitate the storage and retrieval of data. The welding system 18 also provides power to a welding torch, as well as wire and gas through a weld cable 30. In further embodiments, as described in detail below, the welding system 18 may also bidirectionally communicate with the welder 14 via the BCT 10 through either a wired or wireless connection.

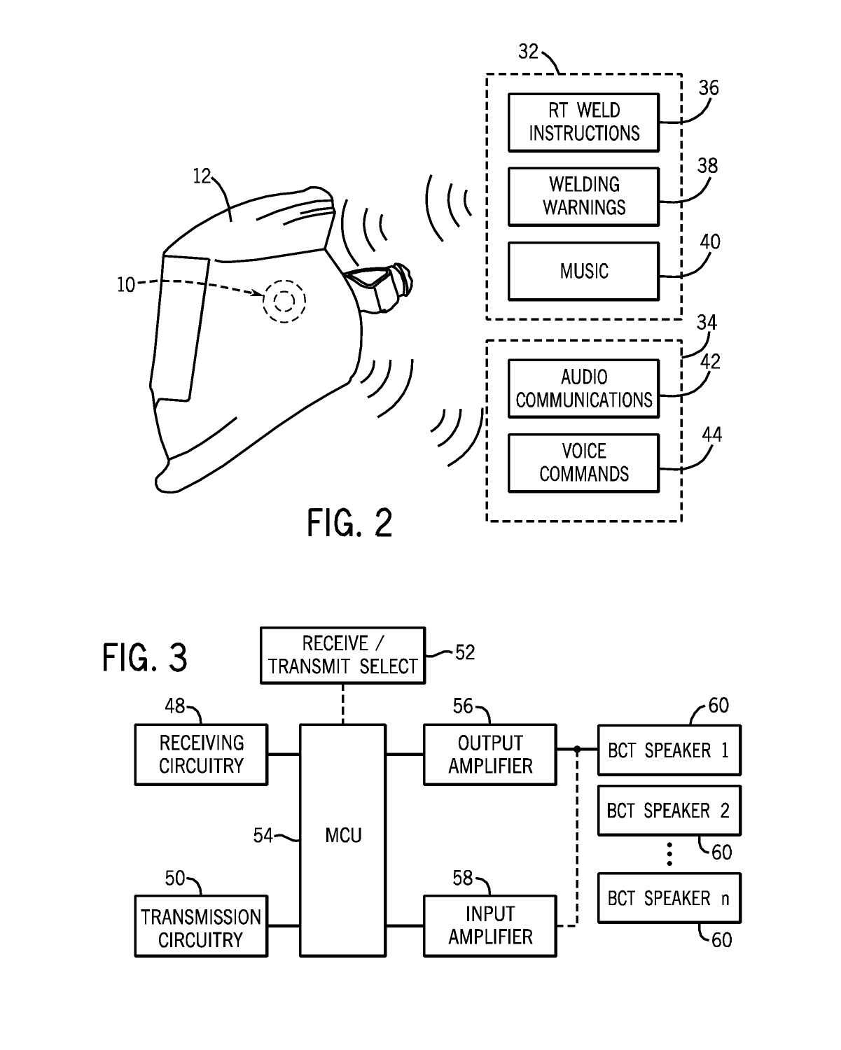

FIG. 2 illustrates a BCT integrated in the welding helmet 12 of FIG. 1 that wirelessly transmits and receives information. That is, the welding helmet 12 may include one or more BCTs configured to function as speakers and/or one or more BCTs (or other devices) configured to function as microphones. The BCTs functioning as microphones may be configured to sense vibrations at the surface of the head of the welder generated by the voice of the welder. Such a feature may have distinct advantages over traditional microphones that sense vibrations in the air after the welder speaks because the vibrations sensed directly from the head of the welder may be less prone to background noise from the welding environment. Nevertheless, in certain embodiments, the BCTs may be used solely as speakers and may be included in systems that employ traditional microphones. Furthermore, the BCTs may be coupled to one or more remote devices or systems, such as a welding power supply controller, via a wireless or wired connection.

In the embodiment illustrated in FIG. 2, the BCTs 10 are located within the welding helmet 12, configured to function as both microphones and speakers, and both receive and transmit data. Accordingly, the welding system of FIG. 2 includes an exemplary set of received data 32 that is transferred to the welding helmet 12 and an exemplary set of transmitted data 34 that is sent from the helmet 12. The received data 32 may include, but is not limited to, real-time weld instructions 36, welding warnings 38, and music 40. That is, in certain embodiments, the BCT 10 may be configured to receive such inputs from a welding system, a stationary or mobile computer, an audio (e.g., music playback device), a telephone, a personal digital assistant) and so forth. For example, the welder may be a student that receives weld instructions 36 via the BCT 10 during a training weld from an instructor or an arc data monitor that monitors characteristics of the student's weld. Furthermore, the BCT 10 may communicate weld warnings (e.g., wire low, torch angle too high or low) from the welding power supply regarding weld parameters. The transmitted data 34 may include audio communications 42 and/or voice commands 44. For example, the welder may utilize the microphone capabilities of the BCT 10 to communicate unidirectionally or bidirectionally with another person. For example, the welder (e.g., a student) may communicate with another welder (e.g., an instructor) via BCTs mounted in their respective helmets. For further example, the welder may communicate with another person via an alternate communication device (e.g., a cell phone, PDA, etc.). In such a way, the welder may utilize the BCTs 10 to bidirectionally or unidirectionally communicate with one or more external devices.

FIG. 3 illustrates exemplary circuitry that may be associated with the BCT in the welding helmet to facilitate the receiving and transmitting of data. In the illustrated embodiment, receiving circuitry 48 is provided to receive and process incoming data. Similarly, transmission circuitry 50 is provided to transmit outgoing signals. A receive/transmit select button 52 may facilitate bidirectional communication between a welder and one or more devices or operators in some embodiments. The receiving circuitry 48, the transmission circuitry 50, and the receive/transmit select button 52 are communicatively coupled to a microcontroller (i.e. MCU) 54 that interfaces system components together, receiving and transmitting various control and processing signals. The MCU 54 is coupled to an output amplifier 56 and an input amplifier 58, which amplify the outgoing and incoming signals, respectively. During transmission, the outgoing amplified signals are transferred to one or more BCTs 60 that transmit the signals to the intended user Likewise, when the BCTs 60 are configured to function as microphones, the incoming signals are routed to the input amplifier 58. In this way, the provided circuitry is configured to both receive and transmit incoming and outgoing signals.

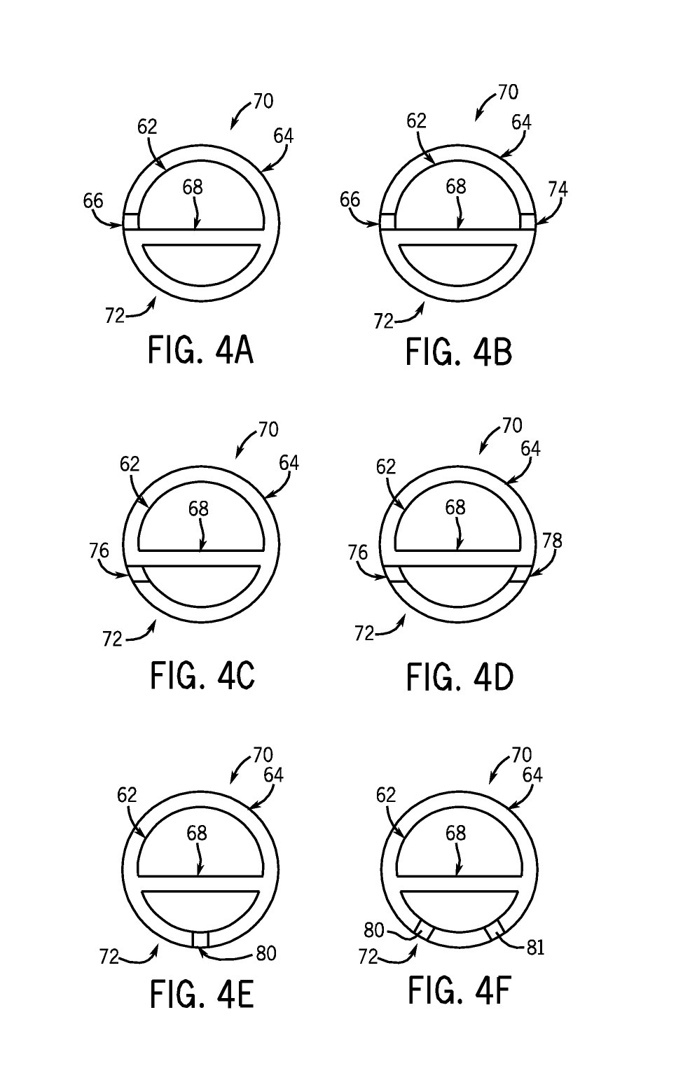

FIGS. 4A-F illustrate a variety of possible placements of one or more BCTs within welding headgear. Specifically, FIG. 4A is a top view of a head of the welder wearing headgear 64. In this embodiment, a single BCT 66 is disposed in front of headband 68 toward a front 70 of the head 62 of the welder and away from a back 72 of the head 62 of the welder. Again, the BCT 66 may be configured to transmit sound directly to the inner ear via bone conduction or sense vibrations from the skull of the welder. In FIG. 4B, BCT 66 is still coupled to the left side of the headgear 64, and a second BCT 74 is coupled to the right side of the headgear 64 in front of the headband 68 toward the front 70 of the head 62 of the welder. In this embodiment, each of the BCTs 66 and 74 may be configured to transmit, receive, or transmit and receive signals.

FIGS. 4C-F illustrate placements of the BCTs behind the headband 68. For example, FIG. 4C is a top view of the welder illustrating a single BCT 76 placed on the left side of the head 62 of the welder behind the headband 68 toward the back 72 of the head 62 of the welder. Again, the BCT 76 may be configured to transmit and/or receive data via bone conduction depending on the specific welding operation. FIG. 4D illustrates a further embodiment of FIG. 4C that includes two BCTs 76 and 78 located on opposite sides of the head 62 of the welder. BCT 78 is coupled to the headgear 64 behind the headband 68 toward the back 72 of the head 62 of the welder. In the embodiment of FIG. 4E, a single BCT 80 is coupled to the headgear 64 at the back 72 of the head 62 of the welder. For example, BCT 80 may be embedded in a comfort cushion located toward the back of the headgear 64. Again, the BCT 80 may be configured to receive and/or transmit signals via bone conduction. Finally, in the embodiment of FIG. 4F, two BCTs 80 and 81 are coupled to the headgear 64 at the back 72 of the head 62 of the welder. It should be noted that in the embodiments of FIG. 4E and FIG. 4F, the one or more BCTs 80 and/or 81 may be attached to the back headband of the headgear 64, located in a comfort cushion, or attached as extensions of the headband.

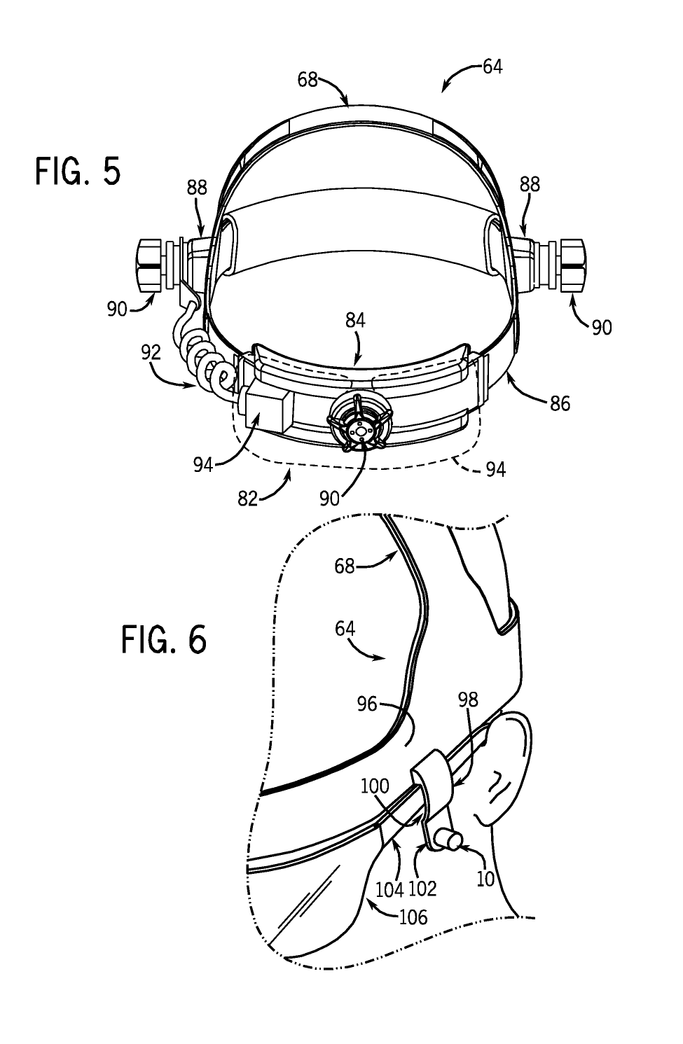

FIG. 5 is a rear perspective view of welding headgear 64 that includes BCT electronics 82 mounted onto a back of the headgear 64 integrated with a comfort cushion 84 that contacts the head of the welder during use. It should be noted that in further embodiments, the electronics 82 may be mounted in a variety of appropriate locations (e.g., anywhere on the headgear 64, in a belt pack, etc.). The headgear 64 includes headband 68 that connects to headband 86 and secures the headgear 64 to the head of the welder during operation. The headgear 64 also includes BCTs 88 mounted below the headband 68 and configured to contact the temples of the welder. One or more adjustment knobs 90 are provided to allow the user to adjust the size of the headgear to fit a variety of head sizes. The BCTs are coupled to the electronics 82 via one or more coiled cords 92 that are configured to expand and contract as the headgear size is adjusted. It should be noted that in other embodiments the cords 92 may be any suitable cords, such as non coiled cords or ribbon cables, which allow adjustment of the headgear size. The electronics 82 may be positioned in any suitable location within dashed line 94. For example, the electronics 82 may be mounted to the back of the headgear 64, molded into the comfort cushion 84 or headband 86, and so forth.

In certain embodiments, the electronics 82 may include a housing 94 that protects the components from damage due to elements (e.g., dirt, weld splatter, etc.) in the surrounding environment. The electronics 82 may also include one or more components that control the operation of the BCTs 88. For example, the one or more components may include controls, such as an audio input (e.g., MP3 player), mute button, volume control, and the like. The components may also include active electronics, passive electronics, electrical components, and so forth. Indeed, the BCTs 88 and their associated circuitry may be active or passive. That is, welding systems may be provided with BCT systems that do not include battery powered components. Still further, in other embodiments, an active BCT system may be provided that includes elements such as amplifiers, power regulators, batteries, photovoltaic cells, wireless radios, transceivers, FM tuners, microcontrollers, light sensors, wireless radio, and so forth. For instance, an audio amplifier may be included in an active system to increase the sound pressure level capabilities, thus rendering the audio signals more audible to the welder in high noise environments.

As previously noted, the BCTs may be mounted to the welding helmet in a variety of suitable locations and ways. FIGS. 6-9 illustrate some of these locations in a variety of adapted welding helmet systems. Specifically, FIG. 6 illustrates headgear 64 including a modified headband 96. In this embodiment, the modified headband 96 includes a flexible extension 98 with an opening 100 and an extension 102. The opening 100 is configured to receive a headband 104 of safety goggles 106. The BCT 10 is mounted to the extension 102 such that the BCT is held firmly against the head of the user. In such embodiments, the BCT may be configured to contact the cheekbone of the user during operation to allow conduction of sound directly to the inner ear. In this way, the BCT may be incorporated into systems with protective eyewear (e.g., safety goggles).

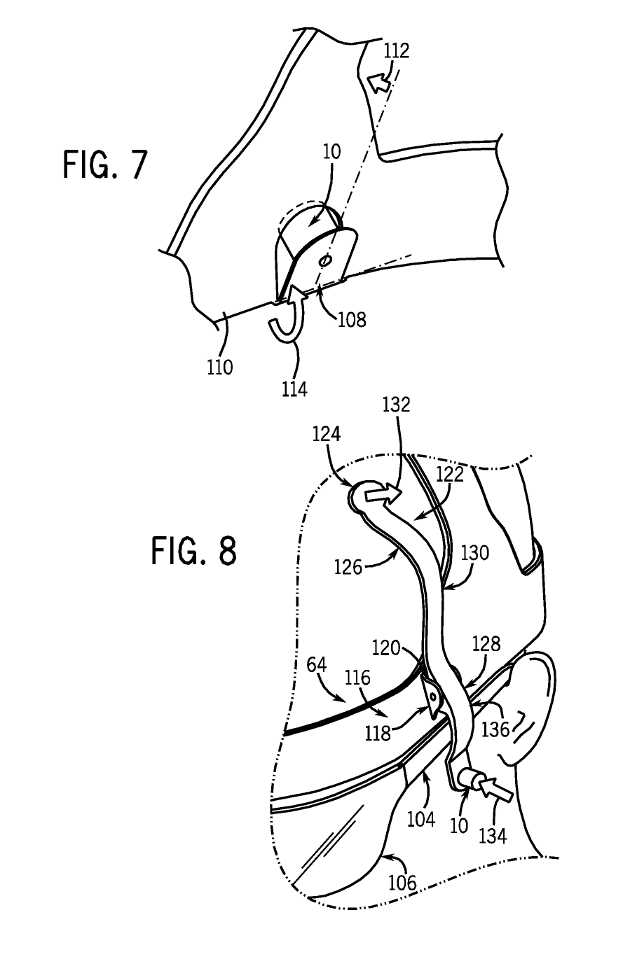

FIG. 7 illustrates an exemplary mechanism for mounting the BCT 10 to the welding headgear 64. In this embodiment, the BCT 10 includes a hinge structure 108 configured to allow outward movement of the BCT 10 when the headgear 64 is placed on the head of the user. In the illustrated embodiment, a base 110 of the headgear 64 is reinforced to accommodate the hinge structure 108. It should be noted that in other embodiments, a spring system may be incorporated into the headgear 64 for use with the hinge structure 108. Additionally, the hinge structure 108 may be located anywhere in the vicinity of the BCT 10. Furthermore, the hinge structure 108 may be molded into the headgear 64 or may be a separate piece that is attachable to the headgear 64. During use, when the headgear 64 is placed on the head of the welder, the head engages the BCT 10, pushing the BCT unit outward, as indicated by arrow 112. This lever action may have the effect of resulting in pressure against the head of the welder. Concurrently, torsion is generated about the reinforced base 110 of the headgear 64, as indicated by arrow 114.

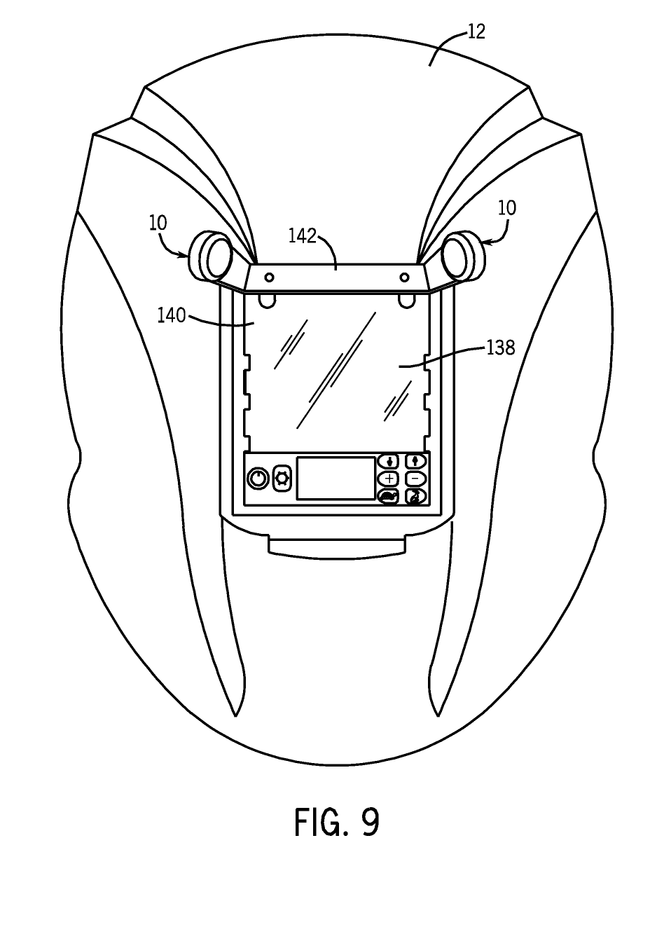

FIG. 8 illustrates another exemplary mechanism for mounting the BCT to the headgear such that the BCT firmly contacts the head of the user during operation. In this embodiment, a modified headband 116 of the headgear 64 includes a torsion spring mechanism 118 disposed at a pivot point 120. A lever 122 includes soft padding 124, a first portion 126, a second portion 128, and the BCT 10. The first portion 126 includes a rounded portion that is configured to extend outward from the head of the user. In some embodiments, the first portion 126 and the second portion 128 of the lever 122 may be made of a suitable flexible material capable of bending during use. The soft padding 124 may be made of any material suitable for buffering the force of the lever 122 against the head to ensure the comfort of the welder.

During operation, when the headgear 64 is placed on the welder, the head of the user presses against the soft pad 124 exerting an outward force, as indicated by arrow 132, and generating a force indicated by arrow 134 at the opposite end of the lever 122. That is, when the user wears the headgear, force 134 holds the BCT 10 firmly against the cheekbone of the user, ensuring that conduction of sound occurs via the cheekbone while bypassing the ear canal. The user may concurrently wear safety goggles 106 without disruption of the placement of the BCT 10 due to an arch 136 in the second portion 128 of the lever 122.

FIG. 9 illustrates an embodiment of the present invention in which the BCTs may be mounted inside the shell of the welding helmet 12. In the illustrated embodiment, a lens 138, which may include a darkening or auto-darkening lens, is mounted to a shell of the welding helmet 12. A lens cartridge 140 is removably mounted inside the welding helmet 12. In the embodiment of FIG. 9, the BCTs 10 are mounted above the lens 138 in the helmet 12 via mounting bar 142. In some embodiments, the BCTs 10 may be mounted to the lens cartridge 140 and may be removable with the cartridge. In other embodiments, the BCTs 10 may be mounted directly to the shell of the helmet 12. Furthermore, the BCTs may be mounted in any suitable position inside the helmet 12. For instance, the BCTs 10 and the mounting bar 142 may be mounted below the lens 138. In such embodiments, the BCTs 10 may be configured to function as speakers that transmit information to the welder.

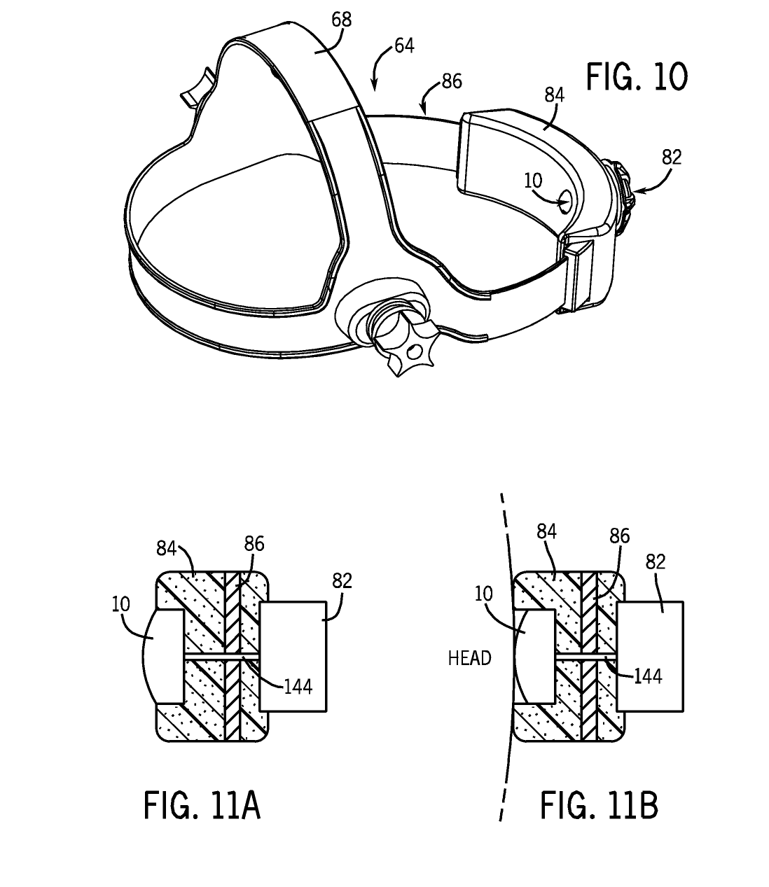

FIG. 10 is a perspective view of another embodiment of the welding headgear 64 including the BCT 10 embedded within the comfort cushion 84 of the headgear 64. In the illustrated embodiment, one BCT 10 is embedded in a center portion of the comfort cushion 84. However, in further embodiments, additional BCTs may be incorporated into the comfort cushion 84. For instance, one BCT may be placed in a left portion of the comfort cushion 84, and one BCT may be placed in a right portion of the comfort cushion. Since the comfort cushion 84 may be made of a suitable material, such as foam, embedding one or more BCTs in the cushion may have the effect of isolating the one or more BCTs from the headband 86. Such a feature may offer distinct advantages since the BCTs may function better in isolation where the BCTs are not prone to ambient noise perturbations. That is, embodiments of the present invention may include one or more BCTs embedded in the comfort cushion of welding headgear (i.e., isolated from ambient noise) and configured to function as speakers.

FIG. 11A and FIG. 11B illustrate cross sectional views of the comfort cushion with an embedded BCT. Specifically, FIG. 11A illustrates the BCT 10 embedded in the comfort cushion 84, thus isolated from the ambient noise in the surrounding environment. The BCT 10 is coupled to the electronics 82 via cable 144. In the embodiment illustrated in FIG. 11A, the headgear is not positioned on a user. Accordingly, a portion of the BCT 10 may extend outward from the comfort cushion 84. The BCT 10 remains embedded in the comfort cushion without impacting the headband 86, thus remaining isolated from the helmet. In FIG. 11B, the comfort cushion 84 is shown positioned against a head during use. As shown, the BCT 10 may become compressed within the comfort cushion 84, thus extending toward the headband 86. However, even during use, the BCT 10 remains isolated in the comfort cushion 84 without contacting the headgear 86. In the embodiments of FIG. 11A and FIG. 11B, the BCT may be configured to function as a speaker, a microphone, or both.

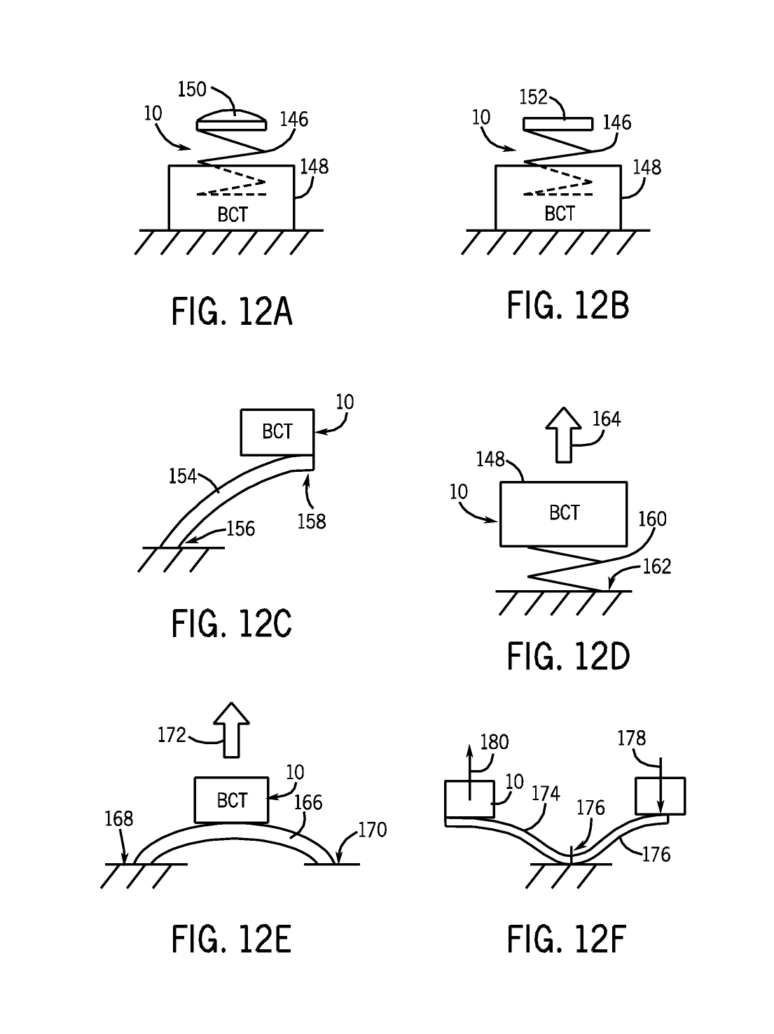

FIGS. 12A-F illustrate a variety of exemplary mechanisms that may be used to ensure that the BCT is held firmly against the head of the user. In FIG. 12A, a spring 146 is disposed within BCT housing 148 and expands outward to contact the user via dome 150 during use. In FIG. 12B, the spring 146 is coupled to a padded disk 152 that is configured to engage the user and transmit sound directly to the inner ear. In FIG. 12C, the BCT 10 is mounted on a lever 154, which includes a fixed end 156 and a movable end 158. The BCT 10 is configured to contact the user by applying pressure at the non-fixed end 158. In FIG. 12D, the BCT 10 and its housing 148 are mounted on a spring 160. The spring 160 includes a fixed end 162 and the spring 160 exerts an outward force, as indicated by arrow 164. During use, such a force ensures that the BCT 10 is held firmly against the head of the user. In FIG. 12E, the BCT is mounted on a lever 166 that includes a first fixed end 168 and a second fixed end 170. During operation, the BCT 10 is pushed outward, as indicated by arrow 172, to contact the head of the user. In FIG. 12F, a first lever portion 174 and a second level portion 176 meet at fixed pivot point 176. As a force is applied to the second lever portion 176, as indicated by arrow 178, the BCT 10 on the first lever portion 174 is forced into contact with the user, as indicated by arrow 180.

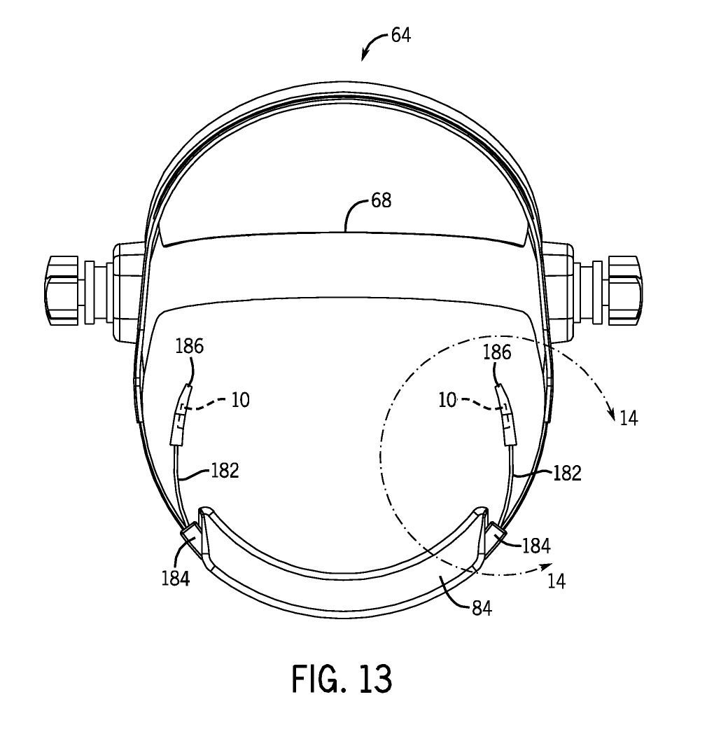

FIG. 13 illustrates a bottom perspective view of another embodiment of the welding headgear 64 with one or more BCTs 10 attached to the headgear via flexible bands 182 made of a suitable material, such as spring tempered stainless steel. In the illustrated embodiment, the bands 182 are adjustably secured into a backplate 184 of the headband that is covered by comfort cushion 84. In certain embodiments, indexing may be provided in the bands 182 and the backplate 184 that facilitate adjustment of the bands 182 to discrete positions. Furthermore, each band (e.g., the left band, the right band, etc.) may be independently adjustable. It should be noted that in further embodiments, the bands 182 may be configured to be received by any section of the headgear 64. For example, the bands 182 may be attached to the overhead section 68 instead of the backplate 184. Indeed, the bands 182 may be attached in any suitable location on the headgear. The bands 182 terminate in BCTs 10 mounted in a suitable cushion material (e.g., foam) 186 that isolates the BCTs from the vibrations of the welding headgear 64. The BCTs 10 may also be covered by a thin membrane that allows audio transmission but protects the BCTs 10 from damage due to particles (e.g., dirt) in the welding environment.

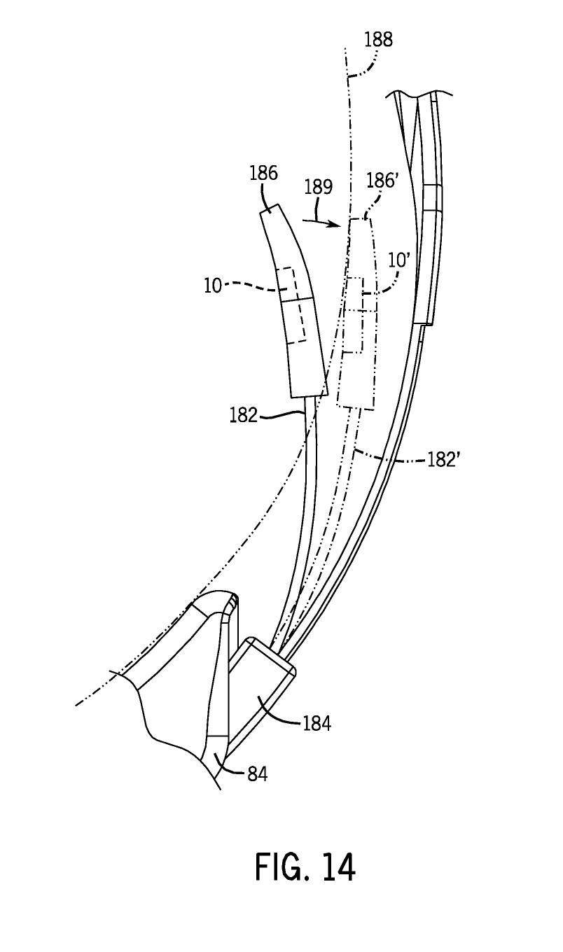

FIG. 14 is a cutaway view of the BCT attachment mechanism of FIG. 13 illustrating the flexibility of the band 182 during use. As previously mentioned, the band 182 may slide in and out of backplate 184 to be adjusted to discrete positions to accommodate a head 188 of the user. Furthermore, the band 182 may be configured to flex as the user places the headgear on the head 188. For example, before placing the headgear on the head 188, the band 182 terminates in cushion 186 with embedded BCT 10 as shown. However, during use, the head 188 of the user may force the band 182 and cushion 186 outward, as indicated by arrow 189, as the headgear is placed on the head 188 of the user. Accordingly, during use, the band 182′, the cushion 186′, and the BCT 10′ have been outwardly displaced to accommodate the head 188. Since the band 182′ remains biased toward the position of band 182, pressure is applied to the head 188 during operation.

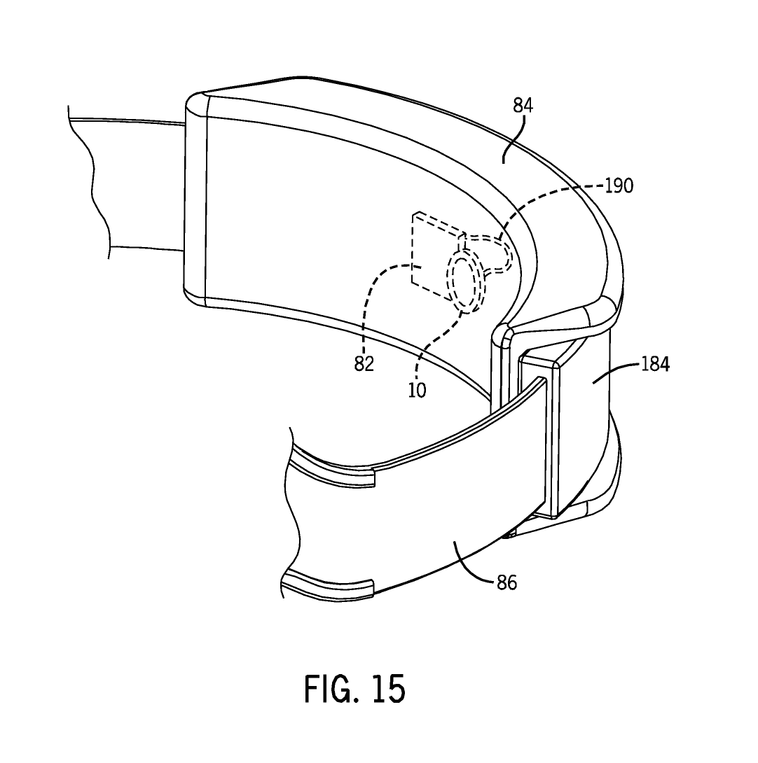

FIG. 15 is a perspective view of an embodiment of a removable accessory (e.g., a comfort cushion) for a welding helmet that includes an embedded BCT and associated electronics. That is, in presently contemplated embodiments, a BCT and its associated electronics may be mounted in a removably securable accessory that may be removed and reattached from the welding helmet. Such a device may be advantageous since the welding operator may remove the accessory, which includes the BCT and its electronics, from one helmet and reattach it to another helmet. This feature may allow more than one welding operator to use the accessory and may allow one operator to use the same accessory with different helmets. Furthermore, this feature may facilitate the replacement or repair of the welding accessory. Specifically, in the illustrated embodiment, BCT 10 and electronics 82 are embedded in the welding accessory (e.g., comfort cushion 84) that is removably secured to the backplate 184 of the headband 86. The BCT 10 and the electronics 82 are coupled via cable 190. As before, the integrated BCT may be configured to receive audio signals from one or more bones of a welding operator via bone conduction (i.e., function as a microphone). The integrated BCT 10 may also be configured to facilitate the transmission of sound directly to an inner ear of the welding operator (i.e., function as a speaker).

While only certain features of the invention have been illustrated and described herein, many modifications and changes will occur to those skilled in the art. It is, therefore, to be understood that the appended claims are intended to cover all such modifications and changes as fall within the true spirit of the invention.

1. A welding helmet, comprising:
a shell;
welding headgear coupled to the shell; and
a bone conduction transducer (BCT) disposed in or on the welding shell and/or the welding headgear to facilitate communication of signals to a welding operator via bone conduction, wherein the bone conduction facilitates the transmission of sound directly to an inner ear of the welding operator.

2. The welding helmet of claim 1, wherein the signals comprise at least one of weld instructions, welding warnings, and music.

3. The welding helmet of claim 1, wherein the welding headgear comprises a headband, and wherein electronics are coupled to the headband and the BCT.

4. The welding helmet of claim 3, wherein the electronics comprise at least one of transmission circuitry, a power source, an amplifier, a power regulator, a transceiver, an FM tuner, a microcontroller, a wireless radio, and a light sensor.

5. The welding helmet of claim 1, wherein the welding headgear comprises a molded extension and an opening configured to receive safety goggles.

6. The welding helmet of claim 1, wherein the welding headgear comprises a reinforced base and a hinge structure, and wherein the BCT is mounted to the hinge structure.

7. The welding helmet of claim 1, wherein the welding headgear comprises one or more levers and a torsion spring mechanism configured to hold the BCT against a cheekbone of the welding operator.

8. The welding helmet of claim 1, wherein the BCT is mounted to the shell above a lens and/or below a lens through which the welding operator views a welding operation.

9. The welding helmet of claim 1, comprising a microphone disposed in or on the welding headgear for detecting sounds during a welding operation and for generating electric signals representative of the sounds.

10. The welding helmet of claim 1, wherein the BCT is attached to the welding headgear via adjustable, flexible bands that are configured to be received by a backplate of the headgear, adjusted to positions in the backplate, and/or flexed outward.

11. A system for welding communication, comprising:
welding headgear; and
a bone conduction transducer (BCT) disposed in or on the welding headgear to facilitate communication of signals via bone conduction from a welding operator to one or more external devices or operators, wherein the bone conduction facilitates the transmission of sound from a bone structure of the welding operator to the BCT.

12. The system for welding communication of claim 11, wherein the signals comprise at least one of audio communications and voice commands.

13. The system for welding communication of claim 11, comprising a speaker disposed in or on the welding headgear for transmitting sounds during a welding operation.

14. The system for welding communication of claim 11, wherein the welding headgear comprises a headband, and wherein electronics are coupled to the headband and the BCT.

15. The system for welding communication of claim 14, wherein the electronics comprise at least one of transmission circuitry, a power source, an amplifier, a power regulator, a transceiver, an FM tuner, a microcontroller, wireless radio, and a light sensor.

16. A welding helmet, comprising:
welding headgear comprising a headband; and
a comfort cushion that is removably securable to or integrated with the headband of the welding headgear, wherein a bone conduction transducer (BCT) is embedded in the comfort cushion and configured to receive audio signals from one or more bones of a welding operator via bone conduction and/or to facilitate the transmission of sound directly to an inner ear of the welding operator.

17. The welding helmet of claim 16, wherein the BCT is configured to compress into the comfort cushion when the welding headgear contacts the welding operator.

18. The welding helmet of claim 17, wherein the BCT does not contact the headband during compression.

19. The welding helmet of claim 16, wherein electronics are mounted on the headband and communicatively coupled to the BCT.

20. The welding helmet of claim 19, wherein the electronics comprise at least one of transmission circuitry, a power source, an amplifier, a power regulator, a transceiver, an FM tuner, a microcontroller, a wireless radio, and a light sensor.

21. An accessory for a welding helmet, comprising:
a bone conduction transducer (BCT) integrated with the welding accessory and configured to receive audio signals from one or more bones of a welding operator via bone conduction and/or to facilitate the transmission of sound directly to an inner ear of the welding operator;
electronics integrated with the welding accessory and coupled to the BCT; and
wherein the welding accessory is configured to be removably attached to the welding helmet.

22. The welding accessory of claim 21, wherein the electronics comprise at least one of transmission circuitry, a power source, an amplifier, a power regulator, a transceiver, an FM tuner, a microcontroller, a wireless radio, and a light sensor.

23. The welding accessory of claim 21, wherein the welding accessory comprises a comfort cushion.
